(19) United States
(12) Patent Application Publication
(10) Pub. No.: US 2016/0354229 A1
(43) Pub. Date: Dec. 8, 2016

(54) APPARATUS FOR USE AS A LIMB RESTRAINT

(71) Applicant: Gregory L. Ruff, Chapel Hill, NC (US)

(72) Inventor: Gregory L. Ruff, Chapel Hill, NC (US)

(21) Appl. No.: 15/241,790

(22) Filed: Aug. 19, 2016

Related U.S. Application Data
(63) Continuation of application No. 14/215,628, filed on Mar. 17, 2014, now abandoned.
(60) Provisional application No. 61/793,083, filed on Mar. 15, 2013.

(51) Int. Cl. A61F 5/37

(52) U.S. Cl. CPC A61F 5/3723

(57) ABSTRACT

A wearable limb restraint comprises a resilient flexible cord having a first end and a second end. A magnet is disposed at each of the first end and the second end of the cord. The magnets are configured to removably attach to each other one in order to form a closed loop with the cord.

### CROSS-REFERENCES

This application is a continuation application of U.S. patent application Ser. No. 14/215,628, filed Mar. 17, 2014, which is related to U.S. provisional application No. 61/793,083, filed Mar. 15, 2013, entitled “MAGNETIC BRACELET”, naming Gregory L. Ruff as the inventor, the contents of both of which are incorporated herein by reference.

### BACKGROUND

An apparatus for use as a limb restraint is described and, more particularly, a limb restraint which can be easily and quickly removed if necessary.

Typically, ropes, canvas, leather straps and buckles, or other means have been used to restrain limbs from movement. Occasionally, the user may find it necessary to quickly remove the restraint to regain comfort or control or even in an emergency. It has been found that conventional restraints are not easily removable. In addition, many prior restraints are not easily adjustable, especially with regard to the tightness of the restraint about the limb.

### SUMMARY

A wearable limb restraint comprises a resilient flexible cord having a first end and a second end. A magnet is disposed at each of the first end and the second end of the cord. The magnets are configured to removably attach to each other one in order to form a closed loop with the cord.

In one aspect, at least one of the magnets is adapted to attach to a magnetically attractable surface.

In another aspect, the magnets may comprise rare earth, neodymium, or magnetic therapy magnets. The magnets may be round, spherical or cylindrical magnets.

In yet another aspect, the magnets are fixedly coupled to the cord. The magnets can be heat sealed into place or held into place by a flexible adhesive, stitching or metal encapsulation. The magnets may be fully contained within the ends of the cord.

In still another aspect, the limb restraint is configured to fit around a neck, one or more wrists, or one or more ankles of a user. The limb restraint is configured to couple to one or more additional limb restraints. The coupling may be removable.

### BRIEF DESCRIPTION OF THE DRAWINGS

For a more complete understanding of the present invention, reference should now be had to the embodiment shown in the accompanying drawings and described below. In the drawings:

FIG. 1 is an elevation view of an embodiment of an apparatus for use as a limb restraint.

FIG. 2 is a cross-section of the apparatus for use as a limb restraint taken along line 2-2 of FIG. 1.

FIG. 3 is an exploded perspective view of the apparatus for use as a limb restraint as shown in FIG. 1.

FIG. 4 is a top perspective view of the apparatus for use as a limb restraint shown in FIG. 1 in place on the wrists of a user.

### DESCRIPTION

Referring now to the drawings, wherein like reference numerals indicate corresponding or similar elements throughout the several views, an embodiment of an apparatus for use as a limb restraint is shown and generally designated at 10. The limb restraint 10 comprises a flexible, elastic cord 12 and magnets 14 disposed at each end of the cord 12. The cord is an elongated flexible, elastic member. The cord may be hollow. In one embodiment, the cord 12 is surgical tubing and the magnets 14 are mounted inside the ends of the tubing. Alternatively, in some embodiments, the cord may be an elongated rectangular flexible member fabricated from, for example, a cloth especially suitable for comfortable placement against the skin. It is understood that the cord may be fabricated of other flexible, elastic materials that are suitable to provide a secure and comfortable fit about the limb of the user.

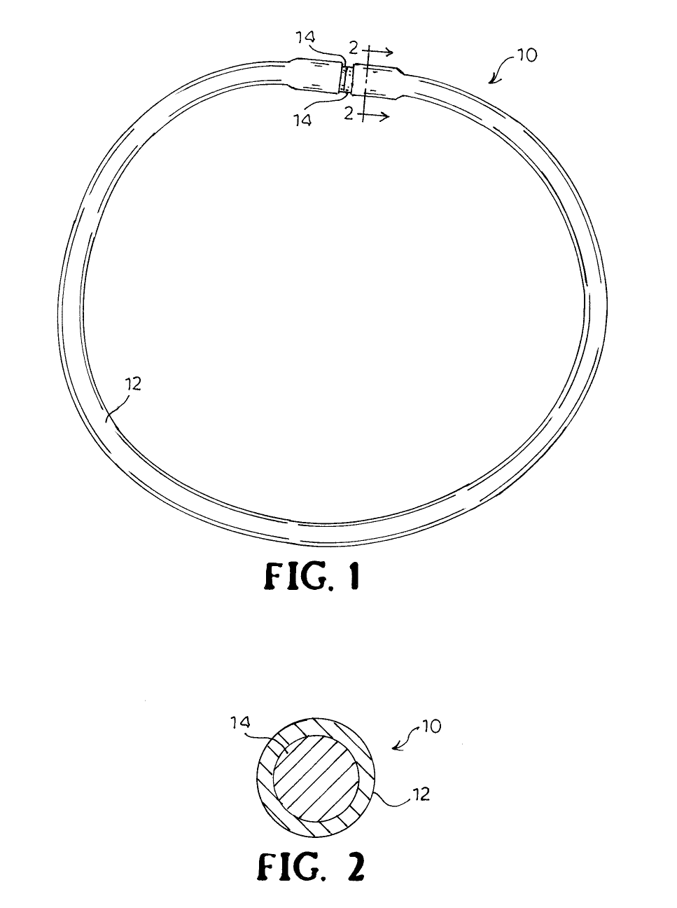

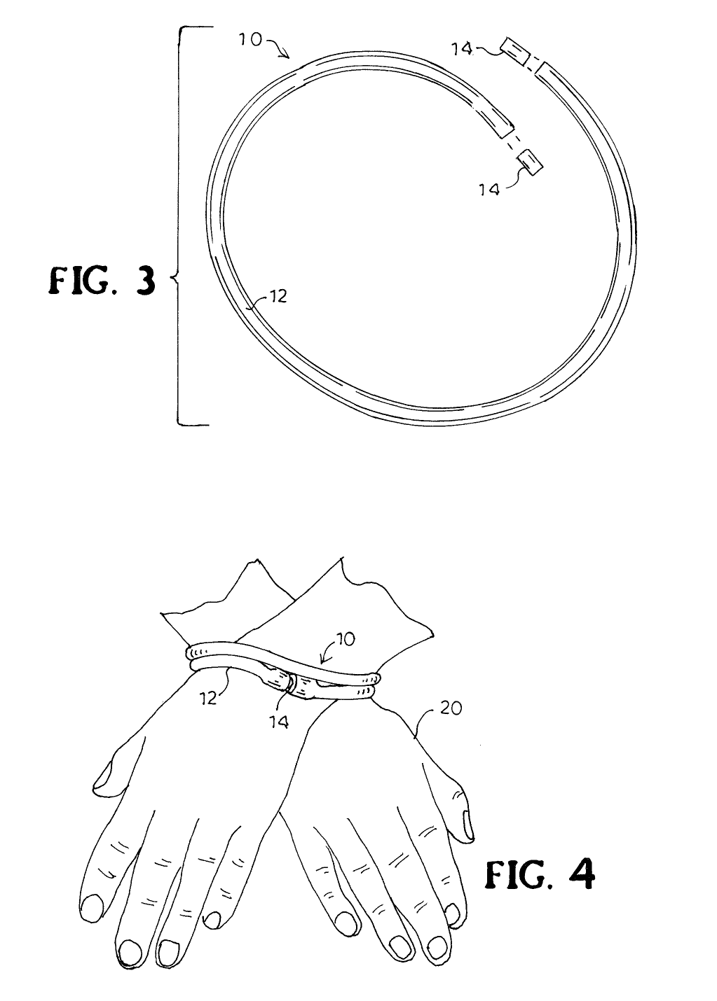

The magnets may comprise rare earth, neodymium, or magnetic therapy magnets. Additionally, the magnets are able to comprise round, spherical or cylindrical magnets. As shown in the FIGS. 1 and 2, the magnets are enclosed within an end of the cord. In some embodiments, the magnets are fixedly coupled to the cord. For example, in some embodiments, the magnets are heat sealed into place. In further embodiments, the magnets are held into place by a flexible adhesive, stitching and/or metal encapsulation. For example, in some embodiments, the magnets are held in place by a flexible silicon adhesive and synthetic rubber with the ability to fix the magnets to a nylon fiber. Alternatively, in some embodiments, the magnets comprise an aperture in the center of the magnets and are strung together through the hole with a cord or string.

The magnets function to removably couple to each other. Specifically, the magnets comprise a north polarity and a south polarity. The magnets are disposed at the ends of the cord such that opposite poles attract each other and enable the magnets to couple together. In this arrangement, the magnets form a closed loop with the cord and secure the wearable limb restraint around a body part of a user or an object. In another, the magnets may connect to a magnetically attractable surface. In some embodiments, the magnetically attractable surface comprises ferrous metal. However, as will be apparent to someone of ordinary skill in the art, the magnetically attractable surface is able to comprise any appropriate magnetically attractable surface.

It is understood that the restraint may be any length, including wherein two or more cords are coupled together in order to create a longer restraint. This is especially useful wherein a length of the cord is used to attach the restraint to a support structure. In this embodiment, a plurality of restraints may be joined end to end in varying configurations in order to increase the length of the restraint. The restraint may then be attached to a support structure by routing the distal end of the cord around a portion of the support structure and pulling to the desired degree of tightness. A loop is formed around the support structure and the magnets are then joined to secure the restraining device to the support structure.

In use, the cord is wrapped around a body part of a user, or an object, and the cord secured by removably coupling the magnets. One or more of the magnets may also be removably coupled with a magnetically attractable object in order to form a closed loop with the cord. FIG. 4 shows the apparatus as it may be used to restrain the wrists of a user. The restraint may be wrapped peripherally around the limb which is to be restrained, such as the wrist or both wrists, of a user to hold the wrists together. A pulling force is exerted on the cord as it is wrapped. Because the cord is flexible and elastic, the degree to which the cord may be stretched as the wrists are encircled, which serves to fix the tightness of the encircling cord about the limb. This feature also provides a means for adjusting both the degree of tightness of the restraint about the limb. The magnets are joined.

A particularly advantageous feature of the restraint is that the user may disconnect the magnets and self-release the cord for escaping the bond. Should the person desire to intentionally remove the restraint, the user can exert force tending to move the limbs apart and pull on the cord such that some lengthening will occur. Sufficient lengthening allows the user to disconnect the magnets and unwrap the cord. Accordingly, the restraint can be quickly and easily attached to or removed. In another embodiment, the limb restraint may comprise piezoelectric transducer to provide an electrical shock or a vibration when the user disconnects the magnets to escape the restraint. The shock or vibration may function as a “penalty” for the escape.

It can be seen that the construction of the present device allows it to be quickly and easily attached, removed or adjusted. The present invention provides a restraint which can be quickly released from the limb or the support structure. Because of the flexibility of the cord, there is no substantial restraint of the limb which cannot be adjusted by force of the user. The ability is there to quickly release the restraint from the limb or the support structure to which it is attached. There is no need to obtain a knife or scissors with which to cut the restraint off the limb or from the support structure. The restraint may be is configured to fit around a neck, a wrist, or an ankle of a user.

I claim:

1. A wearable limb restraint comprising:
a resilient flexible cord having a first end and a second end; and
a magnet disposed at each of the first end and the second end of the cord,
wherein the magnets are configured to removably attach to each other one in order to form a closed loop with the cord.

2. The wearable limb restraint of claim 1 wherein at least one of the magnets is adapted to attach to a magnetically attractable surface.

3. The wearable limb restraint of claim 1 wherein the one or more magnets comprise rare earth, neodymium, or magnetic therapy magnets.

4. The wearable limb restraint of claim 1 wherein the one or more magnets comprise round, spherical or cylindrical magnets.

5. The wearable limb restraint of claim 1 wherein the one or more magnets are fixedly coupled to the cord.

6. The wearable limb restraint of claim 5 wherein the magnets are heat sealed into place.

7. The wearable limb restraint of claim 5 wherein the magnets are held into place by a flexible adhesive, stitching or metal encapsulation.

8. The wearable limb restraint of claim 1 wherein the limb restraint is configured to fit around a neck, one or more wrists, or one or more ankles of a user.

9. The wearable limb restraint of claim 1 wherein the wearable limb restraint is configured to couple to one or more additional limb restraints.

10. The wearable limb restraint of claim 9 wherein the wearable limb restraint is removably coupled with the one or more additional limb restraints.

11. The wearable limb restraint of claim 1 wherein the magnets are fully contained within the ends of the cord.
